(19) United States
(12) Patent Application Publication
(10) Pub. No.: US 2003/0044362 A1
(43) Pub. Date: Mar. 6, 2003

(54) Method and product for skin lightening

(75) Inventors: Robert Edward Gott, Trumbull, CT (US); Jonathan Hague, Rolling Meadows, IL (US); Eng-Beng Lim, Jakarta (ID); Craig Stephen Slavtcheff, Trumbull, CT (US); Alexander Paul Znaiden, Tochigi-ken (JP)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/058,457

(22) Filed: Jan. 28, 2002

(30) Foreign Application Priority Data
Jan. 29, 2001 (GB) 0102235.9

(51) Int. Cl. A61K 7/42; A61K 7/135; A61K 9/70; A61K 31/19

(52) U.S. Cl. 424/59; 424/62; 424/443; 514/557

(57) ABSTRACT

A method and cosmetic product for lightening skin is provided, the method including wiping the skin with a cosmetic towelette. Impregnated on the towelette is an alpha-hydroxy carboxylic acid or salt thereof and a sunscreen agent.

### BACKGROUND OF THE INVENTION

1. Field of the Invention

The invention concerns a method and product for lightening the color of skin.

2. The Related Art

Ever since the first freckle or hyperpigmented spot appeared on the human face, there has been demand for treatment. Historically treatments have involved preparations of mercury, plant extracts and even lemon juice. Bleaching of skin with ammoniated mercury and other salts of this metal are reported to be quite effective. Of course there are significant safety issues involved with mercurials.

Zinc peroxide has been utilized in anhydrous ointments as a bleaching agent. Monobenzyl ether of hydroquinone was marketed for its skin lightening effect but questions of safety were here also raised.

Ascorbic acid preparations, either pure or made from some natural material, such as lemon juice, were suggested as useful. While seemingly entirely safe, they do not seem to be very effective.

U.S. Pat. No. 4,096,240 (Mathur) refers to niacin as effective in skin lightening. This material is postulated to operate by retarding melanin dispersion or distribution into the epidermis. Since unpleasant skin flushing occurs with niacin, the patent suggests use of niacinamide as a substitute. Compositions based upon niacinamide are effective, but only to a limited extent.

U.S. Pat. No. 5,262,153 (Mishima et al.) discloses the use of lactic acid for lightening the color of skin. While this was a useful advance, there remains a need for systems of even greater efficacy.

Accordingly, it is an object of the present invention to provide a method for lightening skin and a product to accomplish this function which is more efficient than previously known methods and products.

Another object of the present invention is to provide a method for lightening skin and a product to accomplish this function which is particularly effective against age spots, freckles and hyperpigmentation.

These and other objects of the present invention will become more readily apparent from consideration of the following summary and detailed description.

### SUMMARY OF THE INVENTION

A method for lightening skin color is provided which includes wiping skin with a cosmetic towelette which includes:

(a) a water-insoluble substrate;

(b) a cosmetic composition impregnated into the substrate, the composition including:

(i) from about 0.1 to about 20% by weight of an alpha-hydroxy carboxylic acid or salt thereof;

(ii) from about 0.1 to about 20% by weight of a sunscreen agent; and

(iii) a pharmaceutically acceptable carrier.

Furthermore, a skin lightening cosmetic product is provided which includes:

(1) a dispensing package; and

(2) a towelette stored within the package awaiting dispensing, the towelette including:

(a) a water-insoluble substrate; and

(b) a cosmetic composition impregnated into the substrate, the composition including:

(i) from about 0.1 to about 20% by weight of an alpha-hydroxy carboxylic acid or salt thereof;

(ii) from about 0.1 to about 20% by weight of a sunscreen agent; and

(iii) a pharmaceutically acceptable carrier;

(3) instructions provided with the product directing a consumer to apply the towelette across areas of the body and thereby achieve a skin lightening effect.

### DETAILED DESCRIPTION OF THE INVENTION

Now it has been found that towelettes impregnated with an alpha-hydroxy carboxylic acid and a sunscreen agent when wiped over the skin are highly effective at lightening skin color. In contrast to mere application by rub-in of an alpha-hydroxy carboxylic acid in a lotion or cream form, application via a towelette results in a statistically higher level of lightening. Normally it would have been expected that wiping would tend to remove actives from the wiped surface. Yet the actual result was quite the opposite. An enhanced effect was noted through use of the towelette to deliver actives.

Compositions of the present invention will contain a C₁-C₂₀ alpha-hydroxy carboxylic acid or salts thereof; as well as mixtures of these materials. The salts are preferably alkali metal, ammonium and C₁-C₁₂ alkanolammonium salts and mixtures thereof. The term “alpha-hydroxy carboxylic acids” includes not only hydroxy acids but also alpha-ketoacids and related compounds of polymeric forms of hydroxyacid.

Alpha-hydroxy carboxylic acids are organic carboxylic acids in which one hydroxyl group is attached to the alpha carbon adjacent the carboxy group. The generic structure is as follows:

(Ra)(Rb)C(OH)COOH

where Ra and Rb are H, F, Cl, Br, alkyl, aralkyl or aryl group of saturated or unsaturated, isomeric or non-isomeric, straight or branched chain or cyclic form, having 1 to 25 carbon atoms, and in addition Ra and Rb may carry OH, CHO, COOH and alkoxy groups having 1 to 9 carbon atoms. The alpha-hydroxy carboxylic acids may be present as a free acid or in lactone form, or in a salt form with an organic base or an inorganic alkali. The alpha-hydroxy carboxylic acids may exist as stereoisomers as D, L, and DL forms when Ra and Rb are not identical.

Typical alkyl, aralkyl and aryl groups for Ra and Rb include methyl, ethyl, propyl, isopropyl, butyl, pentyl, octyl, lauryl, stearyl, benzyl and phenyl, etc. The alpha-hydroxyacids of the first group may be sub-divided into (1) alkyl alpha-hydroxyacids, (2) aralkyl and aryl alpha-hydroxyacids, (3) polyhydroxy alpha-hydroxyacids, and (4) polycarboxylic alpha-hydroxyacids. The following are representative alpha hydroxyacids in each subgroup.

(1) Alkyl Alpha Hydroxyacids

2-Hydroxyethancic acid (Glycolic acid, hydroxyacetic acid)

2-Hydroxypropanoic acid (Lactic acid)

2-Methyl 2-hydroxypropanoic acid (Methyllactic acid)

2-Hydroxybutanoic acid

2-Hydroxypentanoic acid

2-Hydroxyhexanoic acid

2-Hydroxyheptanoic acid

2-Hydroxyoctanoic acid

2-Hyroxynonanoic acid

2-Hydroxydecanoic acid

2-hydroxyundecanoic acid

2-Hydroxydodecanoic acid (Alpha hydroxylauric acid)

2-Hydroxytetradecanoic acid (Alpha hydroxymyristic acid)

2-Hydroxyhexadecanoic acid (Alpha hydroxypalmitic acid)

2-Hydroxyoctadecanoic acid (Alpha hydroxystearic acid)

2-Hydroxyeicosanoic acid (Alpha hydroxyarachidonic acid)

(2) Aralkyl And Aryl Alpha-Hydroxyacids

2-Phenyl 2-hydroxyethanoic acid (Mandelic acid)

2,2-Diphenyl 2-hydroxyethanoic acid (Benzilic acid)

3-Phenyl 2-hydroxypropanoic acid (Phenyllactic acid)

2-Phenyl 2-methyl 2-hydroxyethanoic acid (Atrolactic acid)

2-(4′-Hydroxyphenyl) 2-hydroxyethanoic acid (4-Hydroxymandelic acid)

2-(4′-Chlorophenyl) 2-hydroxyethanoic acid (4-Chloromandelic acid)

2-(3′-Hydroxy-4′-methoxyphenyl) 2-hydroxyethanoic acid (3-Hydroxy-4-methoxymandelic acid)

2-(4′-Hydroxy-3′-methoxyphenyl acid)

3-(2′-Hydroxyphenyl) 2-hydroxypropanoic acid [3(2′Hydroxyphenyl) lactic acid]

3-(4′-Hydroxyphenyl) 2-hydroxypropanoic acid [3-(4′-Hydroxyphenyl) lactic acid]

2-(3′,4′-Dihydroxyphenyl) 2-hydroxyethanoic acid (3,4-Dihydroxymandelic acid)

(3) Polyhydroxy Alpha-Hydroxyacids

2,3-Dihydroxypropanoic acid (Glyceric acid)

2,3,4-Trihydroxybutanoic acid, Isomers; erythronic acid, threonic acid)

2,3,4,5-Tetrahydroxypentanoic acid (Isomers; ribonic acid, arabinoic acid, xylonic acid, lyxonic acid)

2,3,4,5,6-Pentahydroxyhexanic acid (Isomers; allonic acid, altronic acid, gluconic acid, mannoic acid, gulonic acid, idonic acid, galatconic acid, talonic acid)

2,3,4,5,6,7-Hexahydroxyheptanoic acid (Isomers; glucoheptonic acid, galactoheptonic acid etc.)

(4) Polycarboxylic Alpha-Hydroxyacids

2-Hydroxypropane-1,3-dioic acid (Tartronic acid)

2-Hydroxybutane, 1,4-dioic acid (Malic acid)

2,3-Dihydroxybutane-1,4-dioic acid (Tartaric acid)

2-Hydroxy-2-carboxypentane,1,5-dioic acid (Citric acid)

2,3,4,5-Tetrahydroxyhexane,1-5,dioic acid (Isomers: saccharic acid, mucic acid)

(5) Lactone Forms

The typical lactone forms are gluconolactone, galactonolactone, glucuronolactone, glacturonolactone, ribonolactone, saccharic acid lactone, pantoyllactone, glucoheptonolactone, mannonolactone, and galactoheptonolactone.

Representative alpha ketoacids useful for the present invention are as follows.

2-Ketoethanoic acid (Glyoxylic acid)

Methyl 2-ketoethanoate

2-Ketopropanoic acid (Pyruvic acid)

Methyl 2-ketopropanoate (Methyl pyruvate)

Ethyl 2-ketopropanoate (Ethyl pyruvate)

Propyl 2ketopropanoate (Propyl pyruvate)

2-Phenyl-2-ketoethanoic acid (Benzoylformic acid)

Methyl 2-phenyl-2-ketoethanoate (Methyl benzoylformate)

Ethyl 2-phenyl-2-ketoethanoate (Ethyl benzoylformate)

3-Phenyl-2-ketopropanoic acid (Phenylpyruvic acid)

Methyl 3-phenyl-2-ketopropanoate (Methyl phenylpyruvate)

Ethyl 3-phenyl-2-ketopropanoate (Ethyl phenylpyruvate)

2-Ketobutanoic acid

2-Ketopentanoic acid

2-Ketohexanoic acid

2-Ketoheptanoic acid

2-Ketooctanoic acid

2-Ketododecanoic acid

Methyl 2-ketooctanoate

II. Dimeric and Polymeric Forms of Hydroxyacids

When two or more molecules of hydroxycarboxylic acids either identical or non-identical compounds are reacted chemically to each other, dimeric or polymeric compounds will be formed. Such dimeric and polymeric compounds may be classified into three groups, namely (a) acyclic ester, (b) cyclic ester and (c) miscellaneous dimer and polymer.

Representative acylic esters of hydroxycarboxylic acids useful for the present invention are those found below.

Glycolyl glycollate (Glycolic acid glycollate)

Lactyl lactate (Lactic acid lactate)

Mandelyl mandellate

Atrolactyl atrolactate

Phenyllactyl phenyllactate

Benzilyl benzillate

Glycolyl lactate

Lactyl glycollate

Glycolyl glycolyl glycollate

Lactyl lactyl lactate

Lactyl glycolyl lactate

Glycolyl glycolyl glycolyl glycollate

Lactyl lactyl lactyl lactate

Glycolyl lactyl glycolyl lactyl glycollate

Polyglycolic acid and polylactic acid

Most preferred are glycolic acid, lactic acid, alpha-hydroxycaprylic acid, gluconolactone and combinations thereof.

Amounts of the alpha-hydroxy carboxylic acids may range from about 0.1 to about 20%, preferably from about 0.3 to about 12%, more preferably from about 1 to about 8%, optimally from about 2 to about 8% by weight of the total cosmetic composition.

Sunscreen agents of the present invention will have at least one chromophoric group absorbing within the ultraviolet ranging from 290 to 400 nm. Chromophoric organic sunscreen agents may be divided into the following categories (with specific examples) including: p-Aminobenzoic acid, its salts and its derivatives (ethyl isobutyl, glyceryl esters; p-dimethylaminobenzoic acid); Anthranilates (o-aminobenzoates; methyl, menthyl, phenyl, benzyl, phenylethyl, linalyl, terpinyl, and cyclohexenyl esters); Salicylates (octyl, amyl, phenyl, benzyl, menthyl, glyceryl, and dipropyleneglycol esters); Cinnamic acid derivatives (menthyl and benzyl esters, alpha-phenyl cinnamonitrile; butyl cinnamoyl pyruvate); Dihydroxycinnamic acid derivatives (umbelliferone, methylumbelliferone, methylaceto-umbelliferone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naphtholsulfonates (sodium salts of 2-naphthol-3,6-disulfonic and of 2-naphthol-6,8-disulfonic acids); Dihydroxy-naphthoic acid and its salts; o- and p-Hydroxybiphenyldisulfonates; Coumarin derivatives (7-hydroxy, 7-methyl, 3-phenyl); Diazoles (2-acetyl-3-bromoindazole, phenyl benzoxazole, methyl naphthoxazole, various aryl benzothiazoles); Quinine salts (bisulfate, sulfate, chloride, oleate, and tannate); Quinoline derivatives (8-hydroxyquinoline salts, 2-phenylquinoline); Hydroxy- or methoxy-substituted benzophenones; Uric and vilouric acids; Tannic acid and its derivatives (e.g., hexaethylether); (Butyl carbityl) (6-propyl piperonyl) ether; Hydroquinone; Benzophenones (Oxybenzone, Sulisobenzone, Dioxybenzone, Benzoresorcinol, 2,2′,4,4′-Tetrahydroxybenzophenone, 2,2′-Dihydroxy-4,4′-dimethoxybenzophenone, Octabenzone; 4-Isopropyldibenzoylmethane; Butylmethoxydibenzoylmethane; Etocrylene; and 4-isopropyl-dibenzoylmethane).

Particularly useful are: 2-ethylhexyl p-methoxycinnamate, 4,4′-t-butyl methoxydibenzoylmethane, 2-hydroxy-4-methoxybenzophenone, octyldimethyl p-aminobenzoic acid, digalloyltrioleate, 2,2-dihydroxy-4-methoxybenzophenone, ethyl 4-[bis(hydroxypropyl)]aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl)-5-sulfoniobenzoxazoic acid and mixtures thereof.

Suitable commercially available organic sunscreen agents are those identified under the following table.

TABLE I

| CTFA NAME | TRADE NAME | SUPPLIER |
|---|---|---|
| Benzophenone-3 | UVINUL M-40 | BASF Chemical Co. |
| Benzophenone-4 | UVINUL MS-40 | BASF Chemical Co. |
| Benzophenone-8 | SPECTRA-SORB UV-24 | American Cyanamid |
| DEA-Methoxycinnamate | BERNEL HYDRO | Bernel Chemical |
| Ethyl dihydroxypropyl-PABA | AMERSCREEN P | Amerchol Corp. |
| Glyceryl PABA | NIPA G.M.P.A. | Nipa Labs. |
| Homosalate | KEMESTER HMS | Humko Chemical |
| Menthyl anthranilate | SUNAROME UVA | Felton Worldwide |
| Octocrylene | UVINUL N-539 | BASF Chemical Co. |
| Octyl dimethyl PABA | AMERSCOL | Amerchol Corp. |
| Octyl methoxycinnamate | PARSOL MCX | Givaudan Corp. |
| Octyl salicylate | SUNAROME WMO | Felton Worldwide |
| PABA | PABA | National Starch |
| 2-Phenylbenzimidazole-5-sulphonic acid | EUSOLEX 232 | EM Industries |
| TEA salicylate | SUNAROME W | Felton Worldwide |
| 2-(4-Methylbenzylidene)-camphor | EUSOLEX 6300 | EM Industries |
| Benzophenone-1 | UVINUL 400 | BASF Chemical Co. |
| Benzophenone-2 | UVINUL D-50 | BASF Chemical Co. |
| Benzophenone-6 | UVINUL D-49 | BASF Chemical Co. |
| Benzophenone-12 | UVINUL 408 | BASF Chemical Co. |
| 4-Isopropyl dibenzoyl methane | EUSOLEX 8020 | EM Industries |
| Butyl methoxy dibenzoyl methane | PARSOL 1789 | Givaudan Corp. |
| Etocrylene | UVINUL N-35 | BASF Chemical Co. |

Most preferred are organic sunscreens in liquid form when at ambient (25° C.) temperature. Illustrative is octyl methyoxycinnamate.

A surfactant is preferably present in the cosmetic composition. The surfactant should to stably dispense the sunscreen agent within the carrier. Amounts of the surfactant may range from about 0.1 to about 20%, preferably from about 1 to about 10%, optimally from about 2 to about 6% by weight of the composition.

The surfactant may be selected from the group consisting of anionic, nonionic, cationic and amphoteric actives. Particularly preferred nonionic surfactants are those with a C₁₀-C₂₀ fatty alcohol or acid hydrophobe condensed with from about 2 to about 100 moles of ethylene oxide or propylene oxide per mole of hydrophobe; the C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates and combinations thereof.

Nonionic surfactants are preferred. Particularly effective are combinations of sorbitan esters such as Polysorbate 20 and polyoxyethylene (from 2 to 100 E.O.) fatty alcohols or acids such as PEG 40 hydrogenated castor oil (commercially available as Chremophore® RH-40).

Amounts of the cosmetic composition relative to the water-insoluble substrate may range from about 20:1 to about 1:20, preferably from about 10:1 to about 1:10, more preferably from about 2:1 to about 1:2, optimally about 1:1 by weight.

A pharmaceutically acceptable carrier will be present as a vehicle for impregnating the towelette substrates with alpha-hydroxy carboxylic acid and sunscreen agent. Among suitable carriers are humectants, emollients, hydrocarbons, silicone oils and solvents (such as water). Amounts of the pharmaceutically acceptable carrier may range from about 10% to about 99%, preferably from about 55% to about 95%, optimally from about 75% to about 90% by weight of the impregnated cosmetic composition.

Humectants may be employed as carriers in the present invention. Humectants are normally polyols. Representative polyols include glycerin, diglycerin, polyalkylene glycols and more preferably alkylene polyols and their derivatives including propylene glycol, dipropylene glycol, polypropylene glycol, polyethylene glycol and derivatives thereof, sorbitol, hydroxypropyl sorbitol, hexylene glycol, 1,2-butylene glycol, 1,2,6-hexanetriol, isoprene glycol, 2-methyl-1,3-propanediol, ethoxylated glycerol, propoxylated glycerol and mixtures thereof. Amounts of the humectant may range from about 0.01 to about 30%, preferably from about 0.05 to about 5%, optimally from about 0.1 to 2% by weight of the composition.

Emollients may be formulated as useful carriers. These may be selected from hydrocarbons, silicones, fatty alcohols, synthetic or natural esters and combinations thereof. Amounts of the emollients may range from about 0.01 to about 30%, preferably from about 0.1 to about 10%, optimally from about 0.3 to about 2% by weight of the composition.

Hydrocarbons encompass mineral oil, terpenes (such as squalene) and isoparaffins. Amounts may range from about 0.01 to about 10%, preferably from about 0.1 to about 3% by weight of the composition.

Silicone oils may be employed as carriers. These materials can be categorized as either the volatile or non-volatile variety. The term “volatile” as used herein refers to those materials which have a measurable vapor pressure at ambient temperature. Volatile silicone oils are preferably chosen from cyclic or linear polydimethylsiloxanes containing from about 3 to about 9, preferably from about 4 to about 5, silicon atoms. Linear volatile silicone materials generally have viscosities less than 5 centistokes at 25° C. while cyclic materials typically have viscosities of less than 10 centistokes. Examples of commercially available volatile silicone oils are Dow Corning® 344 and Dow Corning® 345.

Nonvolatile silicone oils include polyalkyl siloxanes, polyalkylaryl siloxanes and polyether siloxane copolymers. The essentially non-volatile polyalkyl siloxanes useful herein include, for example, polydimethyl siloxanes with viscosities of from 5 to about 100,000 centistokes at 25° C. Among the preferred non-volatile silicone carriers useful in the present compositions are the polydimethyl siloxanes having viscosities from about 10 to about 400 centistokes at 25° C.

Silicone copolyols are useful as both carriers and emulsifying materials within the context of the present invention. Particularly preferred are dimethiconols which may be linear or branched with average number molecular weight ranging from about 1,000 to about 1 million, preferably from about 20,000 to about 500,000, optimally from about 40,000 to about 100,000. Dimethiconols may be formulated as microemulsions in which the silicone is at levels ranging from about 1 to about 95%, preferably from about 10 to about 60%, optimally from about 20 to about 40% by weight of the microemulsion ingredient. Pre-formed microemulsions are available from suppliers such as Dow Corning, General Electric, Union Carbide, Wacker Chemie, Shin Etsu, and Toray Silicone Company. Particularly preferred is a linear dimethiconol microemulsion at 25% silicone with a maximum particle size of 40 nm, pH 6.5-8 and surfactant combination of dodecylbenzene sulphonic acid triethanolamine/Laureth-24 available from Dow Corning under the trademark DC 2-1870.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the composition. Most preferred is iodopropynyl butylcarbamate available from Lonza Corporation under the trademarks Glydant Plus and Glycasil L. Preservatives are preferably employed in amounts ranging from about 0.001% to about 2% by weight of the composition.

Compositions of the present invention may further include herbal extracts. Illustrative extracts include Centella Asiatica, Ginseng, Ginko Biloba, Chamomile, Green Tea, Scullcap, Nettle Root, Swertia Japonica, Fennel and Aloe Vera extracts and combinations thereof. Amounts of each of the extracts on an actives basis may range from about 0.00001 to about 1%, preferably from about 0.001 to about 0.5%, optimally from about 0.005 to about 0.2% by weight of the composition.

Minor adjunct ingredients may also be present in the compositions. Among these may be vitamins such as Vitamin E esters, Vitamin C, Vitamin A esters, Panthenol and any of the Vitamin B complexes. Retinoids may be employed including retinol, retinyl linoleate, retinyl acetate, retinoic acid and combinations thereof. Anti-irritant agents may also be present including those of steviosides, alpha-bisabolol and glycyhrizzinate salts. Each vitamin, retinoid or anti-irritant agent may be present in amounts ranging from about 0.0001 to about 1.0%, preferably from about 0.001 to about 0.5%, optimally from about 0.01 to about 0.3% by weight of the composition.

The impregnating cosmetic compositions can exhibit pH properties ranging from pH 2 to 10. A preferred embodiment has pH being relatively low, for instance, a pH from about 2 to about 6.5, preferably from about 2.5 to about 4.5.

Compositions of the present invention preferably have a viscosity ranging from about 1 to about 10,000 cps, preferably from about 5 to about 1,000 cps, optimally from about 5 to about 500 cps as measured with a Brookfield LVT viscometer using spindle 4 at 30 rpm as measured at 25° C.

Another important feature of the present invention is that of a substrate which is a water insoluble substance. By “water insoluble” is meant the substrate does not dissolve in or readily break apart upon immersion in water. A wide variety of materials can be used as the substrate. The following nonlimiting characteristics may be desirable: (I) sufficient wet strength for use, (ii) sufficient abrasivity, (iii) sufficient loft and porosity, (iv) sufficient thickness, (v) appropriate size, and (vi) non-reactive with components of the impregnating composition.

Nonlimiting examples of suitable substrates which meet the above criteria include nonwoven substrates, woven substrates, hydroentangled substrates, air entangled substrates and the like. Preferred embodiments employ nonwoven substrates since they are economical and readily available in a variety of materials. By nonwoven is meant that the layer is comprised of fibers which are not woven into a fabric but rather are formed into a sheet, particularly a tissue. The fibers can either be random (i.e., randomly aligned) or they can be carded (i.e. combed to be oriented in primarily one direction). Furthermore, the nonwoven substrate can be composed of a combination of layers of random and carded fibers.

Nonwoven substrates may be comprised of a variety of materials both natural and synthetic. By natural is meant that the materials are derived from plants, animals, insects or byproducts. By synthetic is meant that the materials are obtained primarily from various man-made materials or from material that is usually a fibrous web comprising any of the common synthetic or natural textile-length fibers, or mixtures thereof.

Nonlimiting examples of natural materials useful in the present invention are silk fibers, keratin fibers and cellulosic fibers. Nonlimiting examples of keratin fibers include those selected from the group consisting of wool fibers, camel hair fibers, and the like. Nonlimiting examples of cellulosic fibers include those selected from the group consisting of wood pulp fibers, cotton fibers, hemp fibers, jute fibers, flax fibers, and mixtures thereof. Wood pulp fibers are preferred while all cotton fibers (e.g. cotton pads) are normally avoided.

Nonlimiting examples of synthetic materials useful in the present invention include those selected from the group consisting of acetate fibers, acrylic fibers, cellulose ester fibers, modacrylic fibers, polyamide fibers, polyester fibers, polyolefin fibers, polyvinyl alcohol fibers, rayon fibers and mixtures thereof. Examples of some of these synthetic materials include acrylics such as Acrilan®, Creslan®, and the acrylonitrile-based fiber, Orion®; cellulose ester fibers such as cellulose acetate, Arnel®, and Acele®; polyamides such as Nylons (e.g., Nylon 6, Nylon 66, Nylon 610 and the like); polyesters such as Fortrel®, Kodel®, and the polyethylene terephthalate fibers, Dacron®; polyolefins such as polypropylene, polyethylene; polyvinyl acetate fibers and mixtures thereof.

Nonwoven substrates made from natural materials consist of webs or sheets most commonly formed on a fine wire screen from a liquid suspension of the fibers.

Substrates made from natural materials useful in the present invention can be obtained from a wide variety of commercial sources. Nonlimiting examples of suitable commercially available paper layers useful herein include Airtex®, an embossed airlaid cellulosic layer having a base weight of about 71 gsy, available from James River Corporation, Green Bay, Wis.; and Walkisoft®, an embossed airlaid cellulosic having a base weight of about 75 gsy, available from Walkisoft U.S.A., Mount Holly, N.C.

Nonwoven substrates made from synthetic materials useful in the present invention can also be obtained from a wide variety of commercial sources. Nonlimiting examples of suitable nonwoven layer materials useful herein include HEF 40-047, an apertured hydroentangled material containing about 50% rayon and 50% polyester, and having a basis weight of about 43 grams per square yard (gsy), available from Veratec, Inc., Walpole, Mass.; HEF 140-102, an apertured hydroentangled material containing about 50% rayon and 50% polyester, and having a basis weight of about 56 gsy, available from Veratec, Inc., Walpole, Mass.; Novenet® 149-191, a thermo-bonded grid patterned material containing about 69% rayon, about 25% polypropylene, and about 6% cotton, and having a basis weight of about 100 gsy, available from Veratec, Inc., Walpole, Mass.; HEF Nubtex® 149-801, a nubbed, apertured hydroentangled material, containing about 100% polyester, and having a basis weight of about 70 gsy, available from Veratec, Inc. Walpole, Mass.; Keybak® 951V, a dry formed apertured material, containing about 75% rayon, about 25% acrylic fibers, and having a basis weight of about 43 gsy, available from Chicopee Corporation, New Brunswick, N.J.; Keybak® 1368, an apertured material, containing about 75% rayon, about 5% polyester, and having a basis weight of about 39 gsy, available from Chicopee Corporation, New Brunswick, N.J.; Duralace® 1236, an apertured, hydroentangled material, containing about 100% rayon, and having a basis weight from about 40 gsy to about 115 gsy, available from Chicopee Corporation, New Brunswick, N.J.; Duralace® 5904, an apertured, hydroentangled material, containing about 100% polyester, and having a basis weight from about 40 gsy to about 115 gsy, available from Chicopee Corporation, New Brunswick, N.J.; Sontaro® 8868, a hydroentangled material, containing about 50% cellulose and about 50% polyester, and having a basis weight of about 60 gsy, available from Dupont Chemical Corp.

Most preferred as a towelette for purposes of this invention are non-woven substrates, especially blends of rayon/polyester and ratios of 10:90 to 90:10, preferably ratios of 20:80 to 80:20, optimally 40:60 to 60:40 by weight. A most useful towelette is a 70:30 rayon/polyester non-woven wipe article.

The substrate can be made into a wide variety of shapes and forms. Generally the substrate is in single use towelette form. Advantageously, the towelettes are folded in a Z-shaped formation. They may be interleaved with one another but preferably are not interleaved. The Z fold consists of a center panel flanked by upper and lower wing panels. The upper and lower wing panels are substantially of equal width and substantially half of a width of the center panel. Each towelette is folded medially in a direction orthogonal to that of the Z-shaped formation. Advantageously the size of the towelette may range in length from 10 to 40 cm, preferably from 15 to 30 cm, optimally from 18 to 24 cm. The width of the towelette may range from 8 to 30 cm, preferably from 10 to 25 cm, optimally from 15 to 20 cm.

Anywhere from 5 to 100, preferably from 10 to 50 single towelettes may be stored within a dispensing pouch, preferably a moisture impermeable pouch. During storage and between dispensing, the pouch is resealable, usually via an adhesive strip covering a dispensing opening. Single towelette containing pouches may also be employed.

The substrates of the present invention can comprise two or more layers, each having a different texture and abrasiveness. The differing textures can result from the use of different combinations of materials or from the use of a substrate having a more abrasive side for exfoliation and a softer, absorbent side for gentle cleansing. In addition, separate layers of the substrate can be manufactured to have different colors, thereby helping the user to further distinguish the surfaces.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. The term “body” is meant to denote all areas of the human body including but not limited to the torso, face and limbs.

The following examples will more fully illustrate the embodiments of this invention. All parts, percentages and proportions referred to herein and in the appended claims are by weight unless otherwise illustrated.

### EXAMPLE 1

A composition typical of impregnated fluids of the present invention was formulated in the following manner. Table I lists the components of the composition. Phase A (water) was added to Phase B with continuous mixing until uniformity obtained. Phase C was then folded into the mixture. Components of Phase D were, one by one, added into the combined Phase A, B and C. The resultant composition was heated to 45° C. Components of Phase E were mixed together with vigorous agitation for 3-4 minutes, while heating to 45° C. Phase E was then added to the combined Phase A, B, C and D under moderate agitation. The Brookfield LVT Viscosity of the composition was about 5 cps.

An amount of 4 grams of the composition was impregnated into a polyester/rayon towelette (1.8 gram weight; 6 inch by 8 inch size).

TABLE I

| INGREDIENT | WEIGHT % |
|---|---|
| PHASE A | |
| Water | Balance |
| PHASE B | |
| Glycolic Acid/Ammonium Glycolate (Neutralized pH 4.0) | 4.00 |
| PHASE C | |
| DC 2-1870 (Dimethicone Microemulsion) | 6.00 |
| PHASE D | |
| Glycerin | 0.01 |
| Sodium Lauroamphoacetate | 2.08 |
| Centella Asiatica Extract | 0.10 |
| Ginseng Extract | 0.10 |
| Green Tea | 0.10 |
| Ginko Biloba Extract | 0.10 |
| Glydant Plus ® Liquid (DMDM Hydantoin - Iodopropynyl | 0.20 |
| butylcarbamate) | |
| PHASE E | |
| Ammonium Glycyrrhizinate | 0.05 |
| Chremophore ® RH-40 (PEG040 Hydrogenated Castor | 0.95 |
| Oil) | |
| Polysorbate 20 | 0.95 |
| Octyl Methoxycinnamate (Parsol ® MCX) | 1.00 |
| Fragrance | 0.15 |
| Vitamin E Acetate | 0.001 |
| Alpha Bisabolol | 0.03 |
| Glycasil L (10% Iodopropynyl Butylcarbamate) | 0.05 |
| Retinol 50C | 0.001 |

### EXAMPLES 2-7

A series of compositions are provided under Table II suitable for impregnation into towelettes according to the present invention.

TABLE II

| | EXAMPLES (WEIGHT %) | | | | | |
|---|---|---|---|---|---|---|
| INGREDIENT | 2 | 3 | 4 | 5 | 6 | 7 |
| Phase A | | | | | | |
| Water | 75.00 | 75.00 | 75.00 | 75.00 | 75.00 | 75.00 |
| Phase B | | | | | | |
| Glycolic Acid/Ammonium Glycolate | 6.00 | — | — | 5.00 | 4.00 | — |
| (Neutralized pH 4.0) | | | | | | |
| Potassium Lactate | — | 6.00 | — | — | — | — |
| Lactic Acid | — | — | 6.00 | — | — | 4.00 |
| Phase C | | | | | | |
| DC 2-1870 (Dimethicone Microemulsion) | 6.00 | 6.00 | 6.00 | 6.00 | 6.00 | 6.00 |
| Phase D | | | | | | |
| Glycerin | 0.01 | 0.01 | 0.01 | 0.01 | 0.01 | 0.01 |
| Sodium Lauroamphoacetate | 2.00 | 1.00 | — | — | — | 1.00 |
| Sodium Cocoyl Isethionate | — | 1.00 | 1.00 | — | — | — |
| Sodium Lauryl Sarcosinate | — | — | — | 1.00 | — | — |
| Sodium Cocoamidopropyl Betaine | — | — | — | — | 1.00 | 1.00 |
| Herbal Extracts | 0.40 | 0.40 | 0.40 | 0.40 | 0.40 | 0.40 |
| Glydant Plus Liquid | 0.20 | 0.20 | 0.20 | 0.20 | 0.20 | 0.20 |
| Phase E | | | | | | |
| Ammonium Glycyrrhizinate | 0.05 | 0.05 | 0.05 | 0.05 | 0.05 | 0.05 |
| PEG-40 Hydrogenated Castor Oil | 0.95 | 0.95 | 0.95 | 0.95 | 0.95 | 0.95 |
| Polysorbate 20 | 0.95 | 0.95 | 0.95 | 0.95 | 0.95 | 0.95 |
| Octyl Methoxycinnamate | 2.00 | 1.00 | — | — | — | — |
| Octocrylene | — | — | 1.00 | — | — | — |
| Benzophenone-3 | — | — | — | 1.00 | — | — |
| Octyl Salicylate | — | — | — | — | 1.00 | — |
| Butyl Methoxy Dibenzoyl Methane | — | 1.00 | — | — | — | — |
| Menthyl Anthranilate | — | — | — | — | — | 1.00 |
| Fragrance | 0.15 | 0.15 | 0.15 | 0.15 | 0.15 | 0.15 |
| Vitamin E Acetate | 0.001 | 0.001 | 0.001 | 0.001 | 0.001 | 0.001 |
| Alpha Bisabolol | 0.03 | 0.03 | 0.03 | 0.03 | 0.03 | 0.03 |
| Glycasil L | 0.05 | 0.05 | 0.05 | 0.05 | 0.05 | 0.05 |
| Retinal 50C | 0.001 | 0.001 | 0.001 | 0.001 | 0.001 | 0.001 |
| Water | Bal. | Bal. | Bal. | Bal. | Bal. | Bal. |

Each of the compositions listed in Table II is impregnated into a hydroentangled web of pulp in towelette form. Six grams of each formulation is applied to a towelette (2.2 gram weight; 6 inch by 8 inch size). Towelettes are then stacked, thirty per stack. These are then sealed within a flexible pouch having an opening covered by an adhesively sealed closure flap.

### EXAMPLE 8

A clinical test was performed to evaluate the efficacy of towelettes of this invention in lightening skin. An 8-week clinical test was conducted on 93 female subjects, aged 18-35, of Indonesian decent and all had skin colour in the range 6-8 on the skin colour grading scale applied to Indonesian skin. They were divided into 3 subgroups as follows: towelette vs no treatment, towelette vs placebo, and control cream vs no treatment. In groups with only one product treatment, half of the subjects applied the product on the left forearm and the other half applied it on the right forearm. In the group with two product treatments, half of the subjects applied one product on the left forearm and other product on the right forearm and the other half applied vice versa.

Subjects were asked to use/apply product on their forearm for both lateral (“outer side” of forearm) and volar (inner elbow to inner wrist) twice daily for 8 weeks. A CR-10 Chromameter was used for objective measurement of skin colour at week 0 (baseline), 2, 4, 6 and 8. Three readings were taken at each site, one site designated on the lateral forearm, 5 cm distal from the elbow, and one site on the volar forearm, 5 cm distal from the elbow flex joint (for both left and right forearm). Table III (a-b) sets forth the results of the readings. Greater difference numbers from the baseline indicate higher efficacy.

The “towelette” employed for the studies was that prepared according to Example 1. The placebo did not contain any alpha-hydroxy acid or other potentially active skin lightening agent; it was merely formulated to have similar viscosity and carrier vehicles. “PADC” is a commercially available anti-aging cream containing 8% alpha-hydroxy acid (i.e. glycolic acid) applied without towelette.

Table IIIa reveals statistically significant positive values for the towelette versus no treatment and indicates a strong skin lightening effect. Comparison of the impregnated towelette against a placebo as shown in Table IIIb also indicates a significant lightening effect for the impregnated towelette. Table IIIc was employed as a positive control to evaluate use of alpha-hydroxy acid formulation by itself without towelette application. As expected, there also was a skin lightening effect but the changes were much less than with the towelette. The advantage with the towelette is even more remarkable because the amount of glycolic acid (4%) in the towelette was half that in PADC (8%).

TABLE IIIa
Towelette vs. No Treatment

| | | | | | Mean | | |
|---|---|---|---|---|---|---|---|
| | | | | | change | | |
| | Treatment | | Mean L* | Mean L* | from | | Significancy |
| Site | Paired | Week | at Week i | at Baseline | baseline | Efficacy | p-value |
| Lateral | Towelette | 2 | 48.8333 | 47.8012 | 1.0321 | 0.9126 | 0.0001 |
| | No Treatment | | 47.9471 | 47.8276 | 0.1195 | | |
| | Towelette | 4 | 48.8184 | 47.8012 | 1.0172 | 0.8333 | 0.0002 |
| | No Treatment | | 48.0115 | 47.8276 | 0.1839 | | |
| | Towelette | 6 | 49.1425 | 47.8012 | 1.3413 | 1.1218 | 9658E−09 |
| | No Treatment | | 48.0471 | 47.8276 | 0.2195 | | |
| | Towelette | 8 | 49.1655 | 47.9845 | 1.1810 | 1.0477 | 1.19E−05 |
| | No Treatment | | 48.1524 | 48.0191 | 0.1333 | | |
| Volar | Towelette | 2 | 54.3989 | 53.8575 | 0.5414 | 0.3368 | 0.0579 |
| | No Treatment | | 54.2862 | 54.0816 | 0.2046 | | |
| | Towelette | 4 | 54.1115 | 53.8575 | 0.2540 | 0.3965 | 0.0155 |
| | No Treatment | | 53.9391 | 54.0816 | −0.1425 | | |
| | Towelette | 6 | 54.4575 | 53.8575 | 0.6000 | 0.4977 | 0.0021 |
| | No Treatment | | 54.1839 | 54.0816 | 0.1023 | | |
| | Towelette | 8 | 54.3357 | 54.0179 | 0.3178 | 0.7285 | 5.20E−05 |
| | No Treatment | | 53.8274 | 54.2381 | −0.4107 | | |

TABLE IIIb
Towelette vs. Placebo

| | | | | | Mean | | |
|---|---|---|---|---|---|---|---|
| | | | | | change | | |
| | Treatment | | Mean L* | Mean L* | from | | Significancy |
| Site | Paired | Week | at Week i | at Baseline | baseline | Efficacy | p-value |
| Lateral | Towelette | 2 | 48.7899 | 47.9808 | 0.8091 | 0.601 | 0.0009 |
| | Placebo | | 48.3515 | 48.1434 | 0.2081 | | |
| | Towelette | 4 | 48.8485 | 47.9808 | 0.8677 | 0.6475 | 1.23E−06 |
| | Placebo | | 48.3636 | 48.1434 | 0.2202 | | |
| | Towelette | 6 | 49.1172 | 47.9808 | 1.1364 | 0.9 | 0.0041 |
| | Placebo | | 48.3798 | 48.1434 | 0.2364 | | |
| | Towelette | 8 | 48.9889 | 47.9808 | 1.0081 | 0.5798 | 0.0003 |
| | Placebo | | 48.5717 | 48.1434 | 0.4283 | | |
| Volar | Towelette | 2 | 54.5202 | 54.0192 | 0.501 | 0.1485 | 0.3916 |
| | Placebo | | 54.1333 | 53.7808 | 0.3525 | | |
| | Towelette | 4 | 54.0566 | 54.0192 | 0.0374 | 0.0657 | 0.7408 |
| | Placebo | | 53.7525 | 53.7808 | −0.0283 | | |
| | Towelette | 6 | 54.3818 | 54.0192 | 0.3626 | 0.0373 | 0.8153 |
| | Placebo | | 54.1061 | 53.7808 | 0.3253 | | |
| | Towelette | 8 | 54.2707 | 54.0192 | 0.2515 | 0.2091 | 0.2719 |
| | Placebo | | 53.8232 | 53.7808 | 0.0424 | | |

TABLE IIIc
PADC vs. No Treatment

| | | | | | Mean | | |
|---|---|---|---|---|---|---|---|
| | | | | | change | | |
| | Treatment | | Mean L* | Mean L* | From | | Significancy |
| Site | Paired | Week | at Week i | at Baseline | baseline | Efficacy | p-value |
| Lateral | PADC | 2 | 49.5929 | 48.9107 | 0.6822 | 0.6477 | 0.0003 |
| | No Treatment | | 48.7952 | 48.7607 | 0.0345 | | |
| | PADC | 4 | 49.5726 | 48.9107 | 0.6619 | 0.8059 | 3.70E−05 |
| | No Treatment | | 48.6167 | 48.7607 | −0.1440 | | |
| | PADC | 6 | 49.775 | 48.9107 | 0.8643 | 0.7393 | 0.0005 |
| | No Treatment | | 48.8857 | 48.7607 | 0.1250 | | |
| | PADC | 8 | 49.3951 | 48.8901 | 0.5050 | 0.7815 | 0.0005 |
| | No Treatment | | 48.4247 | 48.7012 | −0.2765 | | |
| Volar | PADC | 2 | 54.5738 | 54.0893 | 0.4845 | 0.5226 | 0.013 |
| | No Treatment | | 54.2643 | 54.3024 | −0.0381 | | |
| | PADC | 4 | 54.4571 | 54.0893 | 0.3678 | 0.475 | 0.0386 |
| | No Treatment | | 53.9559 | 54.0631 | −0.1072 | | |
| | PADC | 6 | 54.3155 | 54.0893 | 0.2262 | 0.4655 | 0.0474 |
| | No Treatment | | 54.0631 | 54.3024 | −0.2393 | | |
| | PADC | 8 | 54.2111 | 54.1395 | 0.0716 | 0.5345 | 0.0034 |
| | No Treatment | | 53.8630 | 54.3259 | −0.4629 | | |

The foregoing description and examples illustrate selected embodiments of the present invention. In light thereof variations and modifications will be suggested to one skilled in the art, all of which are within the spirit and purview of this invention.

What is claimed is:

1. A method for lightening skin comprising wiping skin with a cosmetic towelette, the towelette comprising:
(a) a water-insoluble substrate;
(b) a cosmetic composition impregnated into the substrate, the composition comprising:
(i) from about 0.1 to about 20% by weight of an alpha-hydroxy carboxylic acid or salts thereof;
(ii) from about 0.1 to about 20% by weight of a sunscreen agent; and
(iii) a pharmaceutically acceptable carrier.

2. The method according to claim 1 wherein the composition has a viscosity ranging from about 5 to about 500 cps.

3. The method according to claim 1 wherein the sunscreen agent is octylmethoxycinnamate.

4. The method according to claim 1 wherein the composition has a pH ranging from about 2 to about 6.5.

5. The method according to claim 1 wherein the alpha-hydroxy carboxylic acid is selected from the group consisting of lactic acid, glycolic acid, gluconolactone and mixtures thereof.

6. A skin lightening product comprising:
(1) a dispensing package; and
(2) a towelette stored within the package awaiting dispensing, the towelette comprising:
(a) a water-insoluble substrate; and
(b) a cosmetic composition impregnated into the substrate, the composition comprising:
(i) from about 0.1 to about 20% by weight of an alpha-hydroxy carboxylic acid or salts thereof;
(ii) from about 0.1 to about 20% by weight of a sunscreen agent; and
(iii) a pharmaceutically acceptable carrier;
(3) instructions provided with the product directing a consumer to apply the towelette across areas of the body and thereby achieve a skin lightening effect.
